(19) United States
(12) Patent Application Publication
(10) Pub. No.: US 2020/0179666 A1
(43) Pub. Date: Jun. 11, 2020

(54) ADAPTER SYSTEM COMPRISING A FRAME, ACTIVE INGREDIENT PAD AND LID

(71) Applicant: LTS LOHMANN THERAPIE-SYSTEME AG, Andernach (DE)

(72) Inventors: Michael HORSTMANN, Neuwied (DE); Roman HADASCHIK, Eisenach (DE); Uwe WORTMANN, Marburg (DE)

(21) Appl. No.: 16/637,382

(22) PCT Filed: Aug. 8, 2018
(86) PCT No.: PCT/DE2018/000233

§ 371 Date: Feb. 7, 2020

(30) Foreign Application Priority Data
Aug. 9, 2017 (DE) DE102017007485.2

(51) Int. Cl. A61F 13/40; A61M 5/158

(52) U.S. Cl. CPC A61M 35/006; A61M 5/158

(57) ABSTRACT

The invention relates to an adapter system comprising a frame and a lid that supports an active ingredient pad and serves to close said frame, the active ingredient pad being inserted into the frame in an operating position. The frame and the lid are interconnected force-fittingly and/or form-fittingly when in the operating position. The present invention makes it possible to position the active ingredient pad with precision.

The invention relates to an adapter system comprising a frame and a cover, carrying an active ingredient pad, for closing the frame, wherein the active ingredient pad is inserted into the frame in an operating position.

A foil-like adapter system is known from WO 2009/047774 A2. For the active ingredient delivery, the active ingredient pad arranged on the cover is inserted into the frame without being positioned.

The problem underlying the present invention is to precisely position the active ingredient pad.

This problem is solved by the features of the main claim. For this purpose, the frame and the cover are connected to one another in a force-fit and/or form-locked manner in the operating position.

Further details of the invention will be apparent from the dependent claims and the following descriptions of schematically illustrated exemplary embodiments.

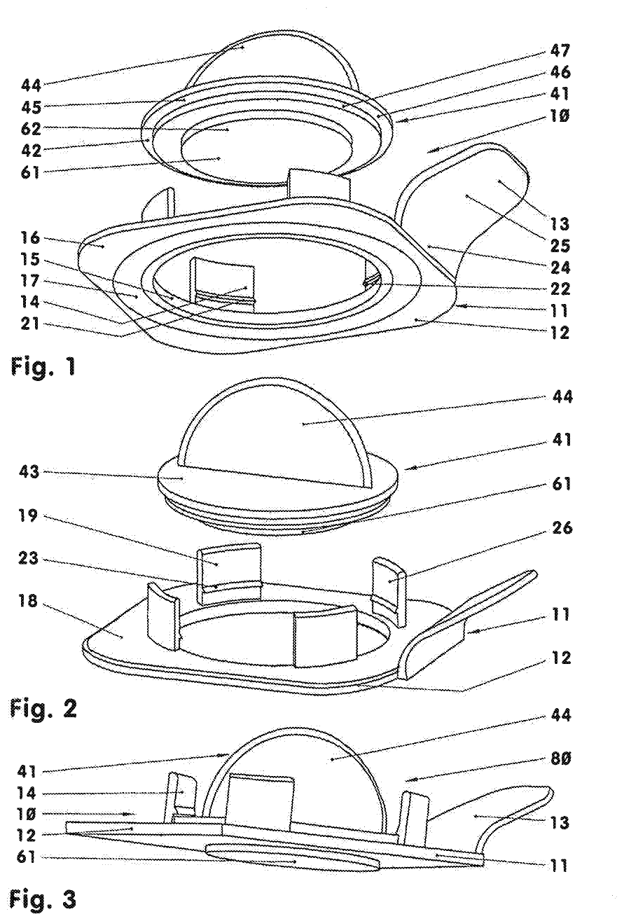

FIG. 1: shows a dimetric view of the adapter system prior to the insertion of the cover;

FIG. 2: shows FIG. 1 from above;

FIG. 3: shows the adapter system with the cover inserted;

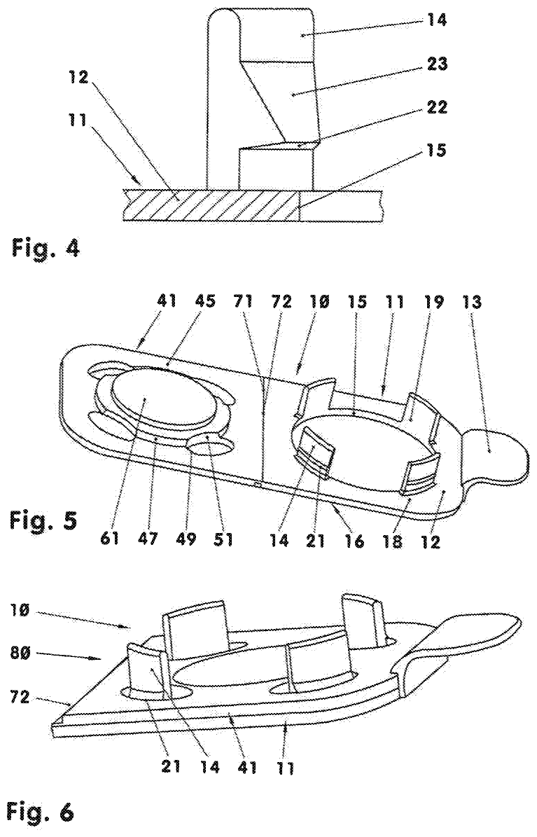

FIG. 4: shows a detail of the frame-side latching engagement;

FIG. 5: shows the adapter system with a pivotable cover;

FIG. 6: shows FIG. 5 with the cover closed;

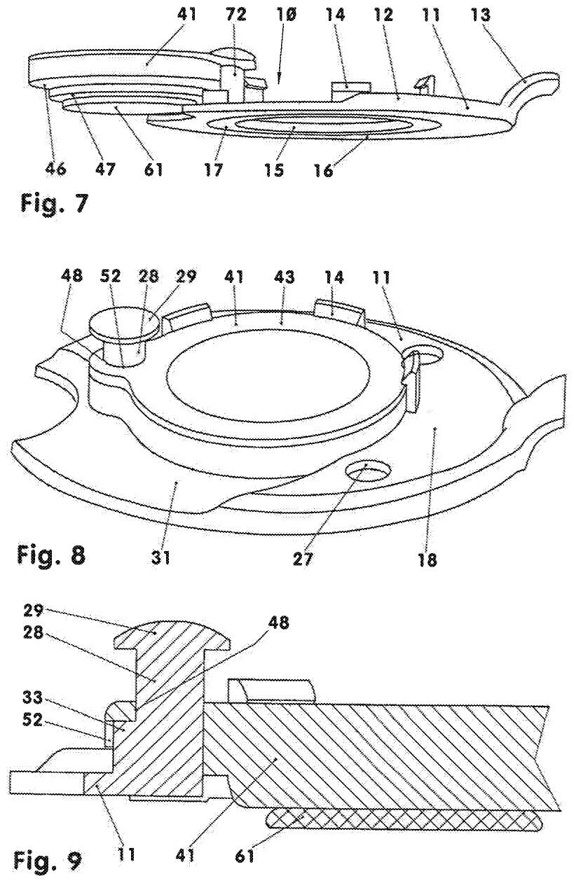

FIG. 7: shows the adapter system with a pivotable cover;

FIG. 8 shows FIG. 7 with the cover closed;

FIG. 9: shows a partial longitudinal sectional view of the adapter system of FIGS. 7 and 8;

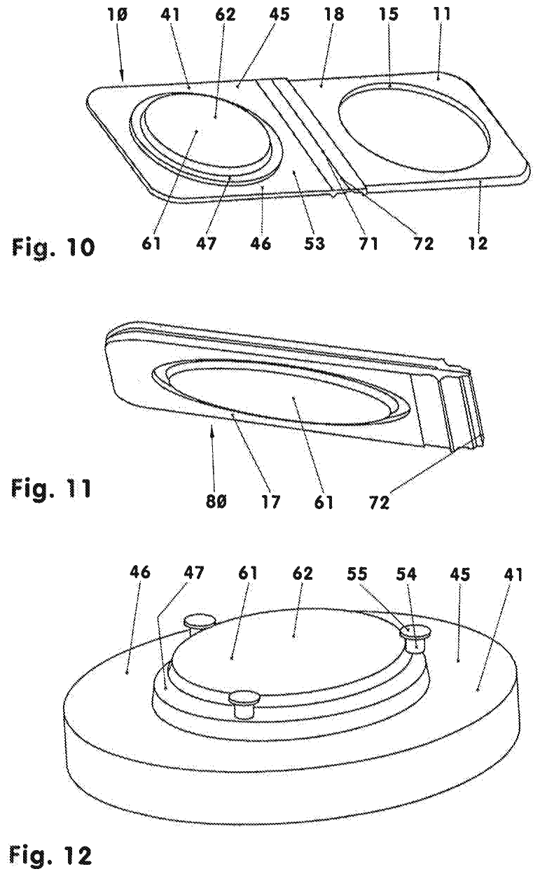

FIG. 10: shows the adapter system with a cover securable in a force-fit manner;

FIG. 11: shows FIG. 10 with the cover closed; and

FIG. 12: shows the securing of the active ingredient pad in the cover.

FIGS. 1 to 4 show an adapter system (10) comprising a frame (11), an active ingredient pad (61) and a cover (41). Such an adapter system (10) is used to deliver active ingredients into the skin of a patient. Since the uppermost layers of the skin, such as the cornea, form a barrier to the active ingredients, these first have to be pretreated. For this purpose, for example, a perforation device is used, which is not shown here and perforates the skin at the site to be treated by means of needles, electrodes or a laser device. After the adapter system (10) has been placed onto the skin, the active ingredients from the active ingredient pad (61) enter the skin through the perforated channels.

In the exemplary embodiment, the frame (11) has an approximately square outline having rounded edges. The frame comprises a support panel (12), a pull tab (13) and, for example, four detent elements (14). An opening (15) having a circular cross-sectional surface, for example, is provided centrally in the support panel (12). The frame (11) is made of a polyolefinic, injection-molded plastic material, for example. This may be LDPE, for example. However, it is also conceivable to produce the frame from other single- or multi-component plastic materials.

An adhesive layer (17) is applied to the bottom side (16) of the support panel (12). This, for example, ring-shaped adhesive layer (17) is initially protected by a protective film, known as a release liner.

The detent elements (14) are arranged on the top side (18) of the support panel (12). These are uniformly distributed around the opening (15), for example, having a distance of two millimeters, for example, from the opening (15). The individual detent element (14), see FIG. 4, has a rod-shaped design and includes a catch lug (21) on the inside (19) thereof. For example, this catch lug (21) includes a detent surface (22), which is provided at least approximately parallel to the top side (18) of the support panel (12), and a guide surface (23). At least approximately here means that the individual detent surface (22) can be inclined as much as 15 degrees relative to a plane parallel to the top side (18), wherein the individual detent surfaces (22) represent sections of a lateral cone surface, the tip of which is located above the top side (18). The individual guide surface (23), for example, includes an angle of 30 degrees with a normal to the support panel. The detent element (14) is designed to be elastically deformable, wherein the upper region thereof can be bent open at least by the amount of the shorter length of the detent surface (22). The detent elements (14) can also be designed as holes, annular grooves, and the like. A design as a hook-and-loop fastener or an adhesive bond is also conceivable.

The pull tap (13) is arranged on one side of the support panel (12) in the exemplary embodiment. It projects from the top side (18) of the support panel (12) in an L-shaped manner, for example, wherein the short leg (24) is integrally formed on the top side (18), and the long leg (25) juts freely to the outside. If necessary, the long leg (25) can include a grip perforation, a roughened area and the like.

The cover (41) comprises a cover panel (42), having a grip piece (44) integrally formed on the top side (43) thereof. The disk-shaped grip piece (44) is oriented normal to the cover panel (42), for example. The bottom side (45) of the cover panel (42) includes an outer contact ring (46) that surrounds a downwardly protruding centering ring (47). The active ingredient pad (61) attached in the cover (41) protrudes from this centering ring (47). This has a circular cross-sectional surface in the top view. The distance between the bottom side (62) of the active ingredient pad (61) and the contact ring (46) in a normal direction to the cover panel (42) is greater than the thickness of support panel (12) of the frame (11), for example. The aforementioned distance can be up to three millimeters greater than the aforementioned thickness, for example. The thickness of the cover panel (42) in the region of the contact ring (46) is slightly smaller, for example, than the distance between the detent surface (22) and the top side (18) of the support panel (12). The difference can be up to two millimeters, for example. The active ingredient pad is initially protected by a protective film.

Prior to use of the perforation device, the adapter system (10) is placed by means of the frame (11) thereof onto the perforation device and centered thereon. Prior to, during or after the perforation of the skin, the protective film is pulled from the adhesive layer (17), and the frame (11) is pressed onto the skin of the patient. The adhesive layer (17) bonds with the skin and positions the frame (11). If the adapter system (10) is affixed to the skin prior to the skin being perforated, it prevents the perforation device from sliding out of place. The perforation device can be removed after the skin has been perforated and the frame (11) has been affixed. The affixed frame (11) now surrounds the pretreated section of the skin, which, for example, is located centrically beneath the opening (15).

Now, after the protective film has been pulled off, the cover (41) is inserted into the frame (11). The cover panel (42) is guided along the guide surfaces (23) in the process, wherein the detent elements (14) are bent open radially outwardly, for example. If necessary, the centering ring (47) is additionally guided by means of the opening (15) during further insertion and/or pressing-in. As soon as the cover panel (42) is located beneath the detent surfaces (22), the detent elements (14) snap back elastically. The detent surfaces (22) are now located above the cover panel (42) and prevent an upward movement of the cover (41). The frame (11) and the cover (41) are now non-detachably connected to one another. The cover (41) is now in an operating position (80).

In the operating position (80), the active ingredient pad (61) introduced into the opening (15) of the frame (11) rests on the skin or is pressed thereon. It may also be spaced apart from the skin by a small amount, such as one millimeter. The pressing force is ensured by means of the form fit of the detent elements (14) and of the cover (41). The active ingredient pad (61) is located exactly above the pretreated surface of the skin, so that all the dispensed active ingredient reaches the lower layers of the skin through the channels introduced into the upper layers of the skin. As a result of the precise positioning, it is not necessary to use an excessive amount of active ingredient. In this way, dosing of the active ingredient which is precisely tailored to the application is possible. It is also possible to match the size of the active ingredient pad (61) precisely to the size of the pretreated area. Even though the pretreated area is very difficult to distinguish from the surrounding, untreated tissue, it is hereby possible to introduce the active ingredient in a precisely dosed and exactly positioned manner.

After an application duration interval, the frame (11) and the cover (41) non-detachably connected thereto can be removed. For example, the frame (11) is pulled on the pull tab (13) for this purpose. In the process, the frame (11), comprising the cover (41) inserted therein, detaches from the skin. If necessary, the active ingredient pad (61) can detach from the cover (41) and remains on the skin. After the frame (11) has been removed, a protective patch can be applied. This optionally then also covers the active ingredient pad (61).

FIGS. 5 and 6 show an adapter system (10) comprising a cover (41) that is pivotable about a pivot joint (72) designed as a living hinge (72). The cover (41) is integrally formed on the frame (11), wherein the transition site (71) is designed as a living hinge (72). It is also conceivable to implement the connection between the cover (41) and the frame (11) by means of an insertable joint. In this exemplary embodiment as well, the adapter system is made of a polyolefinic plastic material, for example using an injection molding process.

The frame (11) has a design similar to the frame (11) of the first exemplary embodiment. It comprises an adhesive ring (17) on the bottom side (16) thereof. The detent elements (14) arranged on the top side (18) of the support panel (12) directly abut the opening (15). The catch lugs (21) are outwardly oriented. The wall thickness and the materials used for the frame (11) correspond to those of the first exemplary embodiment.

In this exemplary embodiment, the cover (41) has an at least approximately square outline. Outside the centering ring (47), four detent openings (49) are provided in the contact surface (46). These have an elliptical outline in the exemplary embodiment and are arranged at regular intervals around the active ingredient pad (61). At the centering ring (47), each of the detent openings (49) cuts out a segment-like section (51).

The active ingredient pad (61) is arranged in the cover (41), as has been described in connection with the first exemplary embodiment.

This adapter system (10) can also be connected to a perforation device, for example. The inner surfaces (19) of the catch hooks (14) are used to center the adapter system (10) on the perforation device. This can, for example, be pushed along the perforation device onto the skin after the protective film has been removed. There, the support frame (11) bonds with the skin by means of the adhesive layer (17), so that the pretreated section of the skin is located within the opening (15).

After the integrated adapter system (10) has been affixed to the skin of the patient and the positioning device has been removed, the cover (41) can be closed. For this purpose, it is pivoted along the living hinge (72) relative to the frame (11). The active ingredient pad (61) is inserted into the frame opening (15). During further pivoting-in, the cover (41) can be centered on the frame (11) by means of the centering ring (47). The catch hooks (14) enter the detent openings (49). The detent elements (14) are elastically deformed to the inside in the process. As soon as the detent surfaces (22) have penetrated the cover (41), they snap back and hold the cover (41) in the closed position. The active ingredient pad (61) and the cover (41) are now in the operating position (80). The active ingredient pad (61) protruding downwardly from the frame (11) can be pressed onto the skin by means of the cover (41).

The active ingredient pad (61) is also precisely positioned above the prepared surface of the skin in this embodiment. As a result of the joint (72) having a single degree of freedom and the form-fit latching engagement in the operating position (80), there is no risk of a faulty operation or faulty positioning of the adapter system (10).

FIGS. 7 to 9 show another adapter system (10) comprising a cover (41) pivotable relative to the frame (11). In this exemplary embodiment as well, the frame (11) comprises a support panel (12) having an opening (15). An adhesive layer (17) is arranged on the bottom side (16) of the support panel (12), The top side (18) of the support panel (12) includes two mutually spaced recesses (27). A pivot pin (28) is arranged on the side of the top side (18) of the frame (11) facing away from the pull tab (13). This is a two-stage pin (28) comprising a rivet-shaped head (29), for example. The head (29) can have been produced by means of a forming process or implemented as a separate part.

The pivot pin (28) has a cylindrical design and includes a guide and stop rib (33) in the lower region thereof abutting the support panel (12).

The shown frame (11) comprises three detent elements (14) on the top side (18) thereof, which are each designed in the same manner as the detent elements (14) shown in FIGS. 1 to 4. These are arranged in a sector of 150 degrees, for example, around the opening (15). A depression (31) is provided on the side of the frame top side (18) facing away from the detent elements (14).

The disk-shaped cover (41) also carries the active ingredient pad (61) in this exemplary embodiment. Moreover, it comprises a centering ring (47) and a contact ring (46) surrounding the same. The cover (41) is seated with a cylindrical pivot bushing (48) in the pivot pin (28). The cover (52) furthermore includes two recess grooves, which are radially offset with respect to one another around the pivot bushing (48) and receive the guide and stop rib (33). The depths of the two recess grooves are different, wherein the deeper recess groove (52) shown in FIG. 9 almost completely receives the guide and stop rib (33). A different design of the pivot joint (72) is also conceivable.

During use, the adapter system (10) is initially affixed to the skin with the cover (41) open. For example, the cover (41) rests with the shorter recess groove on the guide and stop rib (33) and is latchingly engaged therewith. The perforation device is centered in the recesses (27) and perforates the skin in the section delimited by the opening (15). After the perforation device has been removed, the cover (41) is pivoted. During this pivoting, the cover (41) is raised. The active ingredient pad (61) pivots above the depression (31) of the frame top side (18).

As soon as the cover (41) has been pivoted in above the operating position (80), it can be displaced along the pivot pin (28) in the direction of the support panel (12). The cover (41) is guided along the guide and stop rib (33) by means of the recess groove (52) in the process. The active ingredient pad (61) is inserted into the opening (15) of the frame (11). The catch pins (14) are elastically deformed outwardly. As soon as the centering ring (47) has been introduced into the frame (11) and the contact surface (46) of the cover (41) rests on the top side (18) of the frame (11), the detent elements (14) snap back and hold the cover (41) in the operating position (80). If necessary, the cover (41) can additionally be connected to the frame in a force-fit manner, for example by means of adhesive. The active ingredient pad (61) protruding, for example, downwardly from the frame (11) is pressed onto the skin and releases the active ingredient through the skin channels into the lower layers of the skin. The frame (11) is pulled off by means of the pull tab (13), as described above.

FIGS. 10 and 11 show an adapter system (10) in which the frame (11) and the cover (41) are connected to one another by means of a living hinge (72), similarly to the exemplary embodiment of FIGS. 5 and 6. The integrated adapter system (10) shown in FIGS. 10 and 11 is made of a multi-component material. The living hinge (72) is made, for example, of a polyolefinic plastic material, such as polypropylene. The cover (41) and the frame (11) are made of a material having a lower modulus of elasticity than the living hinge (72). This is polyurethane (PUR), ethylene vinyl acetate (EVA) and the like, for example. A large-surface-area adhesive layer (17) is arranged on the bottom side (16) of the support panel (12). Another adhesive layer (53) is applied onto the contact surface (46) of the cover (41).

During use, the adapter system (10) is, for example, initially affixed to the skin with the cover (41) folded open. The elastically deformable material of the frame (11) and of the cover (41) allows the same to conform to the contour of the skin. For the pretreatment of the skin, the perforation device is centered in the opening (15), and the channels are introduced into the skin. Afterwards, the cover (41) can be pivoted about the living hinge (72) in such a way that the active ingredient pad (61) and the centering ring (47) enter the opening (15). The opening (15) of the frame (11) and/or the centering ring (47) of the cover (41) can include insertion tapers. The cover (41) is pushed in, deforming the active ingredient pad (61), until the contact surface (46) rests on the top side (18) of the frame (11). The cover (41) is now bonded to the frame (11) by means of the adhesive layer (53). Moreover, active ingredient from the active ingredient pad (61) is thus released through the channels into the lower layers of the skin. After use, the entire adapter system (10) can be pulled off the skin of the patient.

If necessary, the adapter system (10) can also be adaptable to the perforation device. For this purpose, a guide sleeve can be used, which determines the position of the adapter system (10) relative to the perforation device.

FIG. 12 shows an example of the arrangement of the active ingredient pad (61) in the cover. Three mushroom-shaped pins (54) protruding from the centering ring (47) of the cover (41) are arranged thereon. The mushroom heads (55) engage the active ingredient pad (61), holding it in the position thereof relative to the cover (41).

After the cover (41) is closed, the active ingredient pad (61) bonds with the skin. When the adapter system (10) is pulled off, the active ingredient pad (61) continues to adhere to the skin, whereas the mushroom-shaped pins (54) detach from the active ingredient pad (61). In such an embodiment, no protective patch is necessary for the active ingredient pad (61) after the adapter system (10) has been pulled off, provided the side thereof facing the cover (41) is not adhesive.

Instead of the described mushroom-shaped pins (54), the active ingredient pad (61) can also be held in the position thereof relative to the cover (41) by means of studs, by means of a hook-and-loop fastener or the like. However, the active ingredient pad (61) can also be connected to the cover (41) by means of an adhesive ring. This does not detach until the active ingredient pad (61) has been affixed to the skin.

The described adapter systems (10) can be series-produced. The active ingredient pad (61) can be added in the process in an application-specific manner.

Of course, it is also conceivable to combine the different described embodiments with one another.

### LIST OF REFERENCE NUMERALS

10 adapter system

11 frame

12 support panel

13 pull tab

14 detent elements, catch hooks

15 opening, frame opening

16 bottom side

17 adhesive layer, adhesive ring

18 top side

19 inside, inner surfaces

21 catch lug

22 detent surface

23 guide surface

24 short leg

25 long leg

27 recesses

28 pivot pin

29 head

31 depression

33 guide and stop rib

41 cover

42 cover panel

43 top side

44 grip piece

45 bottom side

46 contact ring, contact surface

47 centering ring

48 pivot bushing

49 detent openings

51 segment-like cut-outs

52 recess groove

53 adhesive layer

54 mushroom-shaped pin, mechanical joining elements

55 mushroom heads

61 active ingredient pad

62 bottom side

71 transition site

72 living hinge, pivot joint

80 operating position

1.-8. (canceled)

9. An adapter system comprising a frame and a cover, carrying an active ingredient pad, for closing the frame, the active ingredient pad being inserted into the frame in an operating position, wherein
in the operating position, the frame and the cover are connected to one another in a force-tit and/or form-locked manner.

10. The adapter system according to claim 9, wherein the cover comprises mechanical connecting elements holding the active ingredient pad.

11. The adapter system according to claim 9, wherein the cover comprises a grip piece.

12. The adapter system according to claim 9, wherein the frame comprises an adhesive layer on the bottom side thereof.

13. The adapter system according to claim 9, wherein the frame comprises a pull tab.

14. The adapter system according to claim 9, wherein the adapter system is arrangeable on a perforation device.

15. The adapter system according to claim 9, wherein the frame and the cover are connected to one another by means of a pivot joint.

16. The adapter system according to claim 9, wherein, in the operating position, the position of the cover with respect to the frame is secured by means of detent elements.
